(19) United States
(12) Patent Application Publication
(10) Pub. No.: US 2024/0358321 A1
(43) Pub. Date: Oct. 31, 2024

(54) METHODS AND SYSTEMS FOR ASSESSING HEMORRHOIDS AND TREATMENT EFFECTIVENESS THEREOF

(71) Applicant: Citius Pharmaceuticals, Inc., Cranford, NJ (US)

(72) Inventors: Ralph R. Turner, Cranford, NJ (US); Alan S. Lader, Cranford, NJ (US); Lisa Duarte, Cranford, NJ (US); Daniel Piacquadio, Cranford, NJ (US)

(21) Appl. No.: 18/648,288

(22) Filed: Apr. 26, 2024

Related U.S. Application Data
(60) Provisional application No. 63/509,247, filed on Jun. 20, 2023.
(60) Provisional application No. 63/498,799, filed on Apr. 27, 2023.

(51) Int. Cl. A61B 5/00; G16H 50/20; G16H 50/50

(52) U.S. Cl. CPC A61B 5/4842; A61B 5/4222; A61B 5/4848; G16H 50/20; G16H 50/50

(57) ABSTRACT

The present disclosure provides methods for assessing the severity of an anorectal disorder, such as hemorrhoids. Also provided herein are systems, including exemplar computer-readable storage media and electronic devices, for performing such methods. Such methods and systems provide effective measures and instruments useful for evaluating treatment effectiveness for patients with anorectal disorders.

### CROSS-REFERENCE TO RELATED APPLICATIONS

The present application claims the benefit of and priority to U.S. Provisional Application No. 63/498,799, filed Apr. 27, 2023 and U.S. Provisional Application No. 63/509,247, filed Jun. 20, 2023, the entire contents of both of which are incorporated herein by reference.

### BACKGROUND

Symptomatic hemorrhoid disease is the fourth leading outpatient gastrointestinal diagnosis and has a considerable impact on quality of life. In the United States, hemorrhoids affect nearly 5% of the population, with approximately 10 million patients annually reporting symptoms. Hemorrhoids are caused by the deterioration of the supporting tissues of the anal cushions, which causes venous dilation and prolapse (protrusion): factors like advancing age, pregnancy, abdominal obesity, and constipation are thought to increase the risk of hemorrhoid development. Symptoms include, for example, pain, burning, itching, swelling, and bleeding.

Hemorrhoids can be categorized into four grades based on the widely used Goligher classification system: (1) Grade I: bleeding but non-prolapsing hemorrhoids; (2) Grade II: hemorrhoids prolapse on straining but reduce spontaneously; (3) Grade III: hemorrhoids prolapse but require manual reduction; and (4) Grade IV: irreducibly prolapsing hemorrhoids. However, such categorizations necessitate medical examination and can be cumbersome and inconvenient for patient reporting purposes.

To facilitate clinical diagnosis and treatment of hemorrhoids, it is therefore of great interest to develop more efficient systems and methods to rate and score the severity of hemorrhoids. In particular, there is a need for effective systems and methods to analyze patient report outcome (PRO) data in assessing effectiveness of a hemorrhoid treatment.

### SUMMARY

The present disclosure is based, at least in part, on the development of a method for assessing the severity of an anorectal disorder, such as hemorrhoids, using patient reported outcomes (PROs), including the intensity and impact of symptoms of pain, burning, itching, swelling, and/or bleeding. Also provided herein are computer-readable storage media and electronic devices for performing such methods. The disclosed methods and systems provide effective measures and indices that are useful for diagnosing and studying patients with hemorrhoidal disease, such as analyzing clinical data to evaluate the effectiveness of a hemorrhoid treatment.

Accordingly, the present disclosure provides a method for assessing an anorectal disorder and treatment effectiveness in a patient, the method comprising: receiving, via an input device, a set of attributes associated with a patient having or suspected having an anorectal disorder; automatically inputting, via a processor, the set of attributes to a model, wherein the model is configured to output a score corresponding to the severity of the anorectal disorder the patient; and outputting the score via an output device.

In some embodiments, the anorectal disorder is selected from the group consisting of a hemorrhoid, an anal wart, an anal fissure, an anorectal abscess, and an anal fistula. In some embodiments, the anorectal disorder is a hemorrhoid. In some embodiments, the hemorrhoid is a Goligher Grade II hemorrhoid. In some embodiments, the hemorrhoid is a Goligher Grade III hemorrhoid.

In some embodiments, the set of attributes comprise one or more symptoms of the patient. In some embodiments, the one or more symptoms comprise pain, burning, itching, swelling, and/or bleeding. In some embodiments, the set of attributes comprise a symptom intensity and/or a symptom impact for the one or more symptoms of the patient. In some embodiments, the symptom intensity is collected daily. In some embodiments, the symptom impact is collected weekly. In some embodiments, the symptom impact comprises a physical summary and/or a mental summary. In some embodiments, the physical summary comprises an anorectal disorder bother and/or a daily activity impact, and wherein the mental summary comprises an emotional impact. In some embodiments, the anorectal disorder comprises a bowel movement interference and/or a bowel pressure, wherein the daily activity impact comprises a normal daily activity impact, an exercise or strenuous activity impact, and/or a sitting impact, and wherein the emotional impact comprises a psychological distress impact, a discomfort impact, and/or a social impact. In some embodiments, the symptom impact comprises discomfort (e.g., mild pain, irritation, tenderness); interference with normal, comfortable bowel movement; pressure in the bowels (e.g., fullness, heaviness); interference with normal daily activities (e.g., tasks that involve walking, standing for extended time, or frequent bending); affecting participation in social activities; difficulty in performing strenuous physical activity or exercise (e.g., lifting or carrying heavy objects, hiking, swimming, golf); difficulty in sitting for long periods of time (e.g., on a hard surface, riding in a vehicle, when working or studying); anxiety; embarrassment; and/or frustration. In some embodiments, the set of attributes comprise one or more demographic factors selected from the group consisting of age, gender, race, ethnicity, income, education, and occupation.

In some embodiments, the set of attributes comprises one or more attributes from a patient reported outcome (PRO), a clinician interview, and/or a cognitive interview.

In some embodiments that may be combined with any of the foregoing, the symptom intensity and/or the symptom impact are represented in a scale having a graduated range of values representing a range of different levels of the symptom intensity and/or the symptom impact. In some embodiments, the scale is a numerical rating scale having a range of numbers corresponding to the levels of severity of the symptom intensity and/or the symptom impact. In some embodiments of the numerical rating scale, a lower number of the scale represents a lower level of severity (e.g., a lower intensity or impact of a symptom, including no symptom) and a higher number of the scale represents a higher level of severity (e.g., a higher intensity or impact, including worst symptom). In some embodiments, the symptom intensity is represented in an 11-point numerical rating scale of 0 to 10, wherein a 0 represents no intensity (no symptom), a 5 represents moderate intensity (moderate symptom), and a 10 represents highest intensity (worst symptom). In some embodiments, the symptom impact is represented in a 5-point numerical rating scale of 0 to 4, wherein a 0 represents no impact and a 4 represents highest impact. In some embodiments, the model is a trained model using a training method selected from the group consisting of 2-class learning classification and regression technique comprising: logistic regression, 2-class SVM, nearest neighbor, decision tree, neural net, random forest, deep learning, support vector regression, linear regression, non-linear regression, multivariate regression, robust regression, neural network regression, random forest regression, and nearest neighbors; a 1-class learning technique comprising: 1-class SVM, minimum enclosing ball, clustering-based anomaly detection, nearest neighbor-based anomaly detection; and a 1.5-class learning technique comprising: joint optimization, transfer, multi-task, and low C-value formulations. In some embodiments, the model has been tested for test-retest reliability and/or internal consistency reliability. In some embodiments, the model has been validated for known-groups validity and/or convergent validity.

In some embodiments that may be combined with any of the foregoing, the symptom intensity and/or the symptom impact scores may be recorded manually by the patients (i.e., in a diary). The manual entries in some embodiments may be later input into an input device by the patient or another person.

In some embodiments that may be combined with any of the foregoing, the output score comprises a symptom intensity score and a symptom impact score. In some embodiments, the symptom intensity score is a daily score. In some embodiments, the symptom impact score is a weekly score. In some embodiments, the symptom impact score comprises a physical summary score and a mental summary score. In some embodiments, the symptom impact score comprises an anorectal disorder bother score, a daily activity impact score, and an emotional impact score.

In some embodiments that may be combined with any of the foregoing, the method further comprises a step of comparing the output score with a predetermined threshold value. In some embodiments, the method further comprises a step of diagnosing the presence and/or severity of the anorectal disorder of the patient based on the output score. In some embodiments, the method further comprises a step of selecting one or more treatment options for the patient based on the output score.

In another aspect, the disclosure provides a method for assessing treatment effectiveness for an anorectal disorder, the method comprising: recording a first set of attributes associated with a patient prior to a treatment and a second set of attributes associated with the patient during or after the treatment, wherein the patient suffers from an anorectal disorder; inputting, via an input device, the first set of attributes associated with the patient prior to a treatment and the second set of attributes associated with the patient during or after the treatment; automatically inputting, via a processor, the first set of attributes and the second set of attributes to a model, wherein the model is configured to output a first score corresponding to the severity of the anorectal disorder of the patient prior to the treatment and a second score corresponding to the severity of the anorectal disorder of the patient during or after the treatment; outputting the first score and the second score, or a representation thereof, via an output device; and determining the effectiveness of the treatment based on the first score and the second score. In some embodiments, the recording of step (a) and inputting of step (b) occur simultaneously. In some embodiments, the recording of step (a) is performed on paper.

In some embodiments, the treatment is determined to be effective treatment for the anorectal disorder if the first score is lower than the second score (i.e., the scores increase with treatment). In some embodiments, the treatment is determined to be effective treatment for the anorectal disorder if the first score is higher than the second score (i.e., the scores lower with treatment). In some embodiments, the first score and the second score are symptom intensity summary (SIS) scores. In some embodiments, the treatment is determined to be effective treatment for the anorectal disorder if the first score is lower than the second score and if the difference between the first score and the second score is greater than a predetermined meaningful change threshold (MCT). In some embodiments, the treatment is determined to be effective treatment for the anorectal disorder if the first score is higher than the second score and if the difference between the first score and the second score is greater than a predetermined meaningful change threshold (MCT).

In yet another aspect, the disclosure provides a method for assessing treatment effectiveness for an anorectal disorder, the method comprising: receiving, via an input device, a first set of attributes associated with a first plurality of patients receiving a first treatment for an anorectal disorder and a second set of attributes associated with a second plurality of patients receiving a second treatment for the anorectal disorder; automatically inputting, via a processor, the first set of attributes and the second set of attributes to a model, wherein the model is configured to output a first score corresponding to the severity of the anorectal disorder for the first plurality of patients and a second score corresponding to the severity of the anorectal disorder for the second plurality of patients; outputting the first score and the second score, or a representation thereof, via an output device; and determining the effectiveness of the first treatment relative to the second treatment based on the first score and the second score.

In some embodiments, the first treatment is determined to be more effective than the second treatment if the first score is higher than the second score. In some embodiments, the second treatment is a control or placebo treatment. In some embodiments, the first treatment is determined to be an effective treatment for the anorectal disorder if the first score is higher than the second score. In some embodiments, the anorectal disorder is selected from the group consisting of a hemorrhoid, an anal wart, an anal fissure, an anorectal abscess, and an anal fistula. In some embodiments, the anorectal disorder is a hemorrhoid. In some embodiments, the hemorrhoid is a Goligher Grade II hemorrhoid. In some embodiments, the hemorrhoid is a Goligher Grade III hemorrhoid.

Further provided herein is a non-transitory computer-readable storage medium storing one or more programs, the one or more programs comprising instructions, which when executed by one or more processors of an electronic device having a display, cause the electronic device to perform the method of any one of the preceding embodiments.

Also provided is an electronic device, comprising: a display; one or more processors; a memory; and one or more programs, wherein the one or more programs are stored in the memory and configured to be executed by the one or more processors, the one or more programs including instructions for performing the method of any one of the preceding embodiments.

While the invention is discussed in the context of exemplified anorectal disorder, it is understood that a skilled person in the art would readily recognize that the invention can be applied to, and is equally useful for, other diseases where patients can report outcome.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 illustrates a diagram of an exemplary process for assessing an anorectal disorder of a patient according to one embodiment of the disclosure.

FIG. 2 illustrates a diagram of an exemplary process for diagnosing and treating an anorectal disorder of a patient according to one embodiment of the disclosure.

FIG. 3 illustrates a diagram of an exemplary process for evaluating treatment for an anorectal disorder of a patient according to one embodiment of the disclosure.

FIG. 4 illustrates a diagram of an exemplary process for evaluating the effect of a treatment for an anorectal disorder on a treatment group versus a control group according to one embodiment of the disclosure.

FIG. 5 illustrates an exemplary electronic device in accordance with some embodiments.

### DETAILED DESCRIPTION OF THE INVENTION

The present disclosure is based, at least in part, on the development of a method for assessing the severity of an anorectal disorder, such as hemorrhoids, using patient reported outcomes, including the intensity and impact of symptoms of pain, burning, itching, swelling, and/or bleeding. Also provided herein are computer-readable storage media and electronic devices for performing such methods. The disclosed methods and systems provide effective measures and indices that are useful for diagnosing and studying patients with hemorrhoidal disease, such as analyzing clinical data to evaluate the effectiveness of a hemorrhoid treatment.

### I. Methods for Assessing Anorectal Disorders

Aspects of the present disclosure relate to methods for assessing anorectal disorder.

In one aspect, the present disclosure provides a method for assessing an anorectal disorder and treatment effectiveness in a patient, the method comprising: receiving, via an input device, a set of attributes associated with a patient having or suspected having an anorectal disorder; automatically inputting, via a processor, the set of attributes to a model, wherein the model is configured to output a score corresponding to the severity of the anorectal disorder the patient; and outputting the score via an output device.

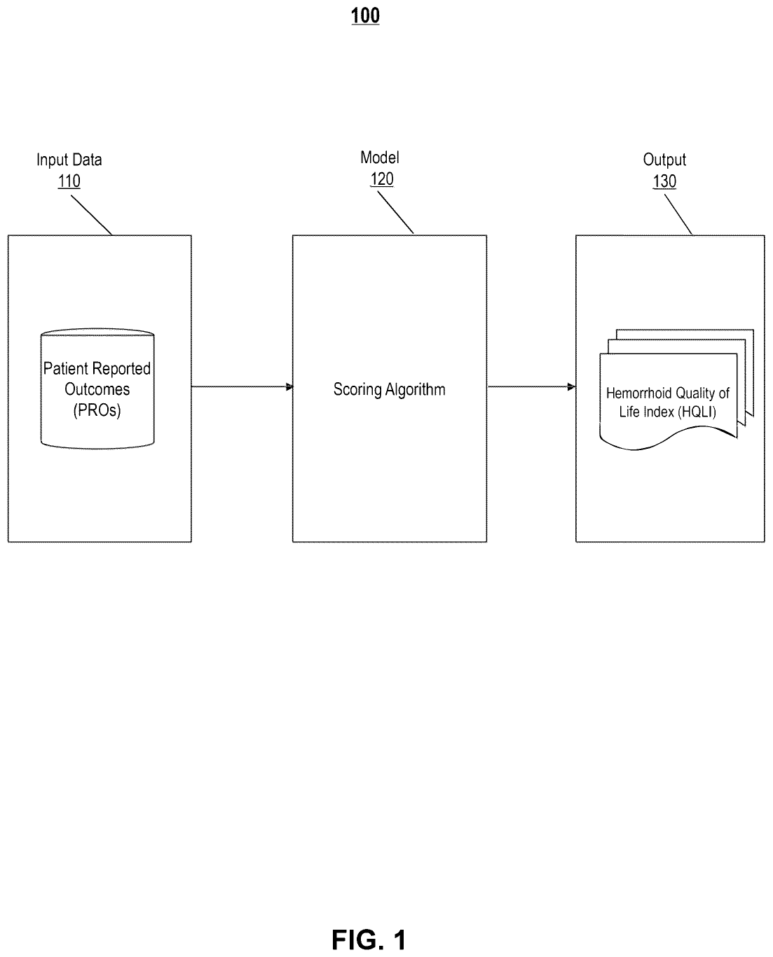

FIG. 1 illustrates a diagram of an exemplary process of such a method for assessing anorectal disorders. Generally, the input data 110 is passed on to the scoring algorithm model 120, which is configured to output a score corresponding to the severity of the anorectal disorder the patient 130. Detailed description of the process is as follows.

While the invention is discussed in the context of exemplified anorectal disorder, it is understood that a skilled person in the art would readily recognize that the invention can be applied to, and is equally useful for, other diseases where patients can report outcome.

### A. Anorectal Disorder and Hemorrhoids

The term “anorectal disorder” includes any disorder associated with an anal rectal disease, including an acute or chronic anal fissure, an internally or externally thrombosed hemorrhoid, a hemorrhoidal disease, a disorder associated with endoscopic hemorrhoidal ligation or pain caused by such ligation, levator spasm, constipation, and other anorectal disorder caused by hypertonicity or spasm of the anal sphincter muscle. The term also refers to post-surgical pain associated with hemorrhoidectomy or other analrectal surgery that leads to intense anal spasms. The term “anal fissure” is also referred to as “anal rhagades” and spasms of the anal sphincter are also referred to as “rectal tenesmus.” Additionally, the term is meant to include pain which can be associated with any of the above disorders or conditions.

In some embodiments, the anorectal disorder is selected from the group consisting of a hemorrhoid, an anal wart, an anal fissure, an anorectal abscess, and an anal fistula. In some embodiments, the anorectal disorder is a hemorrhoid.

As used herein, “hemorrhoids” refer to swelling varicose veins in the mucous membrane inside or just outside the rectum. The composition and methods of the invention may be used to treat diseases of the anorectum which manifest one or more symptoms of itching, discomfort, pain and bleeding.

Hemorrhoids can be categorized into four grades based on the widely used Goligher's classification system: (1) Grade I: bleeding but non-prolapsing hemorrhoids; (2) Grade II: hemorrhoids prolapse on straining but reduce spontaneously; (3) Grade III: hemorrhoids prolapse but require manual reduction; and (4) Grade IV: irreducibly prolapsing hemorrhoids.

In some embodiments, the hemorrhoid is a Goligher Grade II hemorrhoid. In some embodiments, the hemorrhoid is a Goligher Grade III hemorrhoid.

### B. Input of Patient Reported Outcome (PRO) Data

With reference to FIG. 1, input data 110 may contain a set of attributes associated with the patient useful for assessing anorectal disorders. In some embodiments, the set of attributes comprises one or more attributes from a patient reported outcome (PRO), a clinician interview, and/or a cognitive interview.

A patient-reported outcome (PRO) is a health outcome directly reported by the patient who experienced it. It stands in contrast to an outcome reported by someone else, such as a physician-reported outcome, a nurse-reported outcome, and so on. PRO questionnaires may be used in clinical trials or other clinical settings, to help better understand a treatment's efficacy or effectiveness.

In some embodiments of the method, the set of attributes comprise one or more symptoms of the patient. In some embodiments, the one or more symptoms comprise pain, burning, itching, swelling, and/or bleeding. In some embodiments, the set of attributes comprise a symptom intensity and/or a symptom impact for the one or more symptoms of the patient. In some embodiments, the symptom intensity is collected daily. In some embodiments, the symptom impact is collected weekly. In some embodiments, the symptom impact comprises a physical summary and/or a mental summary. In some embodiments, the physical summary comprises an anorectal disorder bother and/or a daily activity impact, and wherein the mental summary comprises an emotional impact.

In some embodiments, the anorectal disorder bother comprises a bowel movement interference and/or a bowel pressure, wherein the daily activity impact comprises a normal daily activity impact, an exercise or strenuous activity impact, and/or a sitting impact, and wherein the emotional impact comprises a psychological distress impact, a discomfort impact, and/or a social impact. In some embodiments, the set of attributes comprise one or more demographic factors selected from the group consisting of age, gender, race, ethnicity, income, education, and occupation.

### C. Model of Scoring Algorithm

With reference to FIG. 1, the model 120 may contain an algorithm that analyzes the input PRO data to assess the symptoms and severity of the anorectal disorder of the patient. In some embodiments, the model is configured to process the input data of 110 to output a score representing the severity of the anorectal disorder the patient 130.

In some embodiments, the symptom intensity and/or the symptom impact are represented in a numerical rating scale. In some embodiments, the symptom intensity is represented in an 11-point numerical rating scale of 0 to 10, wherein a 0 represents no intensity (no symptom), a 5 represents moderate intensity (moderate symptom), and a 10 represents highest intensity (worst symptom). In some embodiments, the symptom impact is represented in a 5-point numerical rating scale of 0 to 4, wherein a 0 represents no impact and a 4 represents highest impact.

In some embodiments, the model is a trained model. In some embodiments, the training method is selected from the group consisting of 2-class learning classification and regression technique comprising: logistic regression, 2-class SVM, nearest neighbor, decision tree, neural net, random forest, deep learning, support vector regression, linear regression, non-linear regression, multivariate regression, robust regression, neural network regression, random forest regression, and nearest neighbors; a 1-class learning technique comprising: 1-class SVM, minimum enclosing ball, clustering-based anomaly detection, nearest neighbor-based anomaly detection; and a 1.5-class learning technique comprising: joint optimization, transfer, multi-task, and low C-value formulations. In some embodiments, the model has been tested for test-retest reliability and/or internal consistency reliability. In some embodiments, the model has been validated for known-groups validity and/or convergent validity.

### D. Output of Hemorrhoid Quality of Life Index (HQLI)

With reference to FIG. 1, the output score 130 represents the severity of the anorectal disorder the patient, produced by the model 120 from scoring the input PRO data 110.

The output score may be represented as an index, such as a Hemorrhoid Quality of Life Index (HQLI). Detailed analysis of the exemplary HQLI and its component items can be found in the Examples section of this application.

In some embodiments that may be combined with any of the foregoing, the output score comprises a symptom intensity score and a symptom impact score. In some embodiments, the symptom intensity score is a daily score. In some embodiments, the symptom impact score is a weekly score. In some embodiments, the symptom impact score comprises a physical summary score and a mental summary score. In some embodiments, the symptom impact score comprises an anorectal disorder bother score, a daily activity impact score, and an emotional impact score.

### II. Methods for Diagnosing and Treating Hemorrhoids

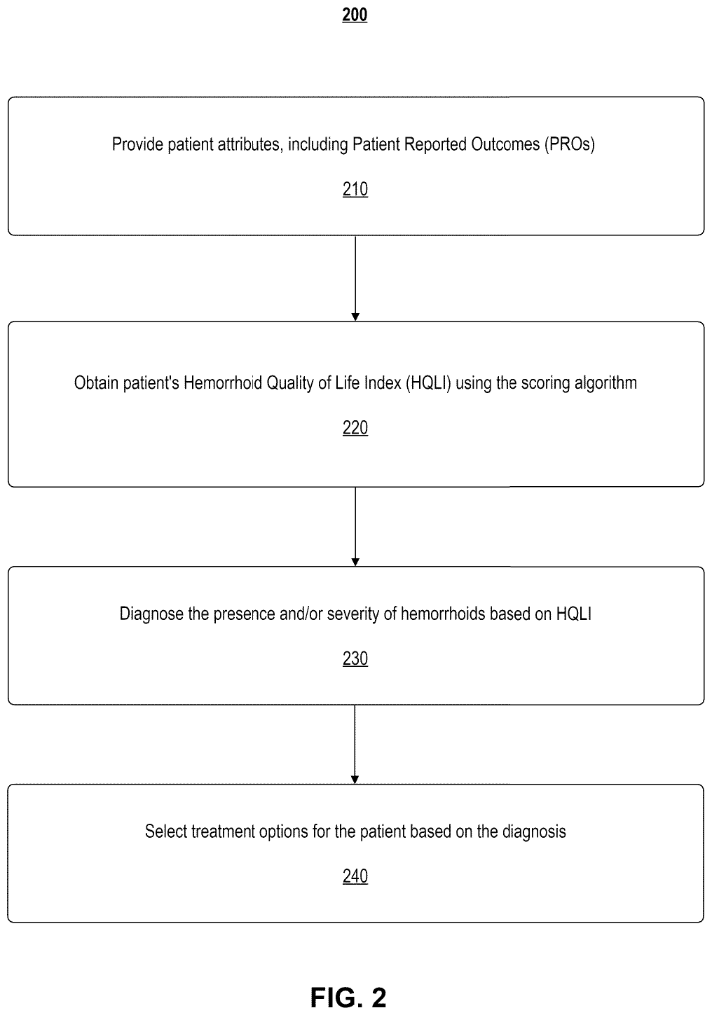

Some aspects of the present disclosure relate to methods for diagnosing and treating hemorrhoids. FIG. 2 illustrates a diagram of an exemplary process for diagnosing and treating an anorectal disorder of a patient according to one embodiment of the disclosure. Generally, the process may include a step 210 providing patient attributes as input, such as using PRO data; a step 220 obtaining output, such as a HQLI using the scoring algorithm; a step 230 diagnosing the presence and/or severity of hemorrhoids based on the output such as the HQLI; and/or a step 240 selecting one or more suitable hemorrhoid treatments based on the diagnosis.

In some embodiments, the method further comprises a step of comparing the output score with a predetermined threshold value. In some embodiments, the method further comprises a step of diagnosing the presence and/or severity of the anorectal disorder of the patient based on the output score. In some embodiments, the method further comprises a step of selecting one or more treatment options for the patient based on the output score.

Methods and techniques for treating anorectal disorders are well known in the art. Various suitable treatment options may be used for the method of the disclosure. As used herein, the term “treating” or “treatment” covers the treatment of the disease or condition of interest in a mammal, preferably a human, having the disease or condition of interest, and includes preventing the disease or condition from occurring in a mammal, in particular, when such mammal is predisposed to the condition but has not yet been diagnosed as having it, inhibiting the disease or condition, i.e., arresting its development, relieving the disease or condition, i.e., causing regression of the disease or condition, or relieving the symptoms resulting from the disease or condition, i.e., relieving pain without addressing the underlying disease or condition. The term “treating” also includes any effect, e.g., lessening, reducing, modulating, or eliminating, that results in the improvement of the condition, disease, disorder and the like.

In some embodiments, the method further comprises administering to the patient an effective amount of a composition comprising a lidocaine, a halobetasol, and a pharmaceutically acceptable carrier. In some embodiments, the lidocaine is lidocaine hydrochloride. In some embodiments, the halobetasol is halobetasol propionate. In some embodiments, the lidocaine is about 3% to about 7% by weight. In some embodiments, the lidocaine is 5% or 5.78% by weight. In some embodiments, the halobetasol is about 0.01% to about 0.06% by weight. In some embodiments, the halobetasol is 0.02% or 0.05% by weight. In some embodiments, the halobetasol is in solubilized state. In some embodiments, the pharmaceutically acceptable carrier comprises a thickener, a solubilizer, an emulsifier, a humectant, a cellulosic polymer, or a combination thereof. In some embodiments, the pharmaceutically acceptable carrier comprises propylene glycol, diethylene glycol monoethylether (DGME), glycerin, hydroxypropyl cellulose, purified water, or a combination thereof. In some embodiments, the composition is in the form of a cream, an ointment, a gel, a lotion, a foam, a sitz bath, a wipe, an aerosol spray, or a liquid. In some embodiments, the composition is in the form of a cream. In some embodiments, administering comprises administering topically to the perirectal area or the anal canal. In some embodiments, administering comprises applying topically twice daily for seven days. In some embodiments, the patient is an adult of 18 years old or older. In some embodiments, the hemorrhoid is a Grade II or Grade III hemorrhoid.

### III. Methods for Evaluating Treatment Effectiveness

Some aspects of the present disclosure relate to methods for evaluating treatment effectiveness.

### A. Evaluating Treatment Efficacy at Different Timepoints

In some aspect, the disclosure provides a method for assessing treatment effectiveness for an anorectal disorder, the method comprising: recording a first set of attributes associated with a patient prior to a treatment and a second set of attributes associated with the patient during or after the treatment, wherein the patient suffers from an anorectal disorder; inputting, via an input device, the first set of attributes associated with the patient prior to a treatment and the second set of attributes associated with the patient during or after the treatment; automatically inputting, via a processor, the first set of attributes and the second set of attributes to a model, wherein the model is configured to output a first score corresponding to the severity of the anorectal disorder of the patient prior to the treatment and a second score corresponding to the severity of the anorectal disorder of the patient during or after the treatment; outputting the first score and the second score, or a representation thereof, via an output device; and determining the effectiveness of the treatment based on the first score and the second score. In some embodiments, the recording of step (a) and inputting of step (b) occur simultaneously. In some embodiments, the recording of step (a) is performed on paper.

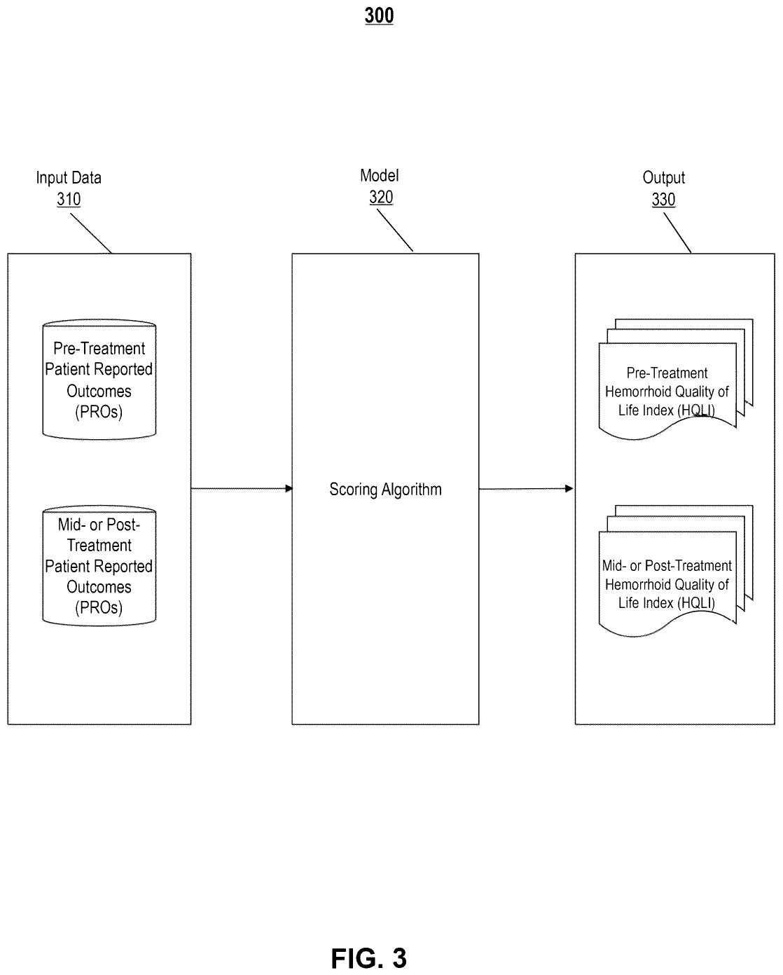

FIG. 3 illustrates a diagram of such an exemplary process for evaluating treatment for an anorectal disorder of a patient according to one embodiment of the disclosure. Generally, the input data 310 may contain a set of pre-treatment patient PRO data as well as a set of mid- or post-treatment patient PRO data; the input is passed onto the scoring algorithm model 320, which is configured to output a score corresponding to the pre-treatment severity of the anorectal disorder the patient as well as a score corresponding to the mid- or post-treatment severity of the anorectal disorder the patient 330. The process may further contain a step of comparing the pre-treatment score with the mid- or post-treatment score to monitor the progress of the symptoms and/or determine the effectiveness of the treatment. In some variations, the mid- or post-treatment patient PRO data may comprise patient PRO data collected from multiple timepoints during the treatment process.

In some embodiments, the treatment is determined to be effective treatment for the anorectal disorder if the first score is lower than the second score (i.e., the scores increase with treatment). In some embodiments, the treatment is determined to be effective treatment for the anorectal disorder if the first score is higher than the second score (i.e., the scores decrease with treatment). In some embodiments, the first score and the second score are symptom intensity summary (SIS) scores. In some embodiments, the treatment is determined to be effective treatment for the anorectal disorder if the first score is lower than the second score and if the difference between the first score and the second score is greater than a predetermined meaningful change threshold (MCT). In some embodiments, the treatment is determined to be effective treatment for the anorectal disorder if the first score is higher than the second score and if the difference between the first score and the second score is greater than a predetermined meaningful change threshold (MCT).

Meaningful change is the ability to measure clinically important change in a clinical state (Liang M H. Longitudinal construct validity: Establishment of clinical meaning in patient evaluative instruments. Medical Care. 2000; 38:Ii 84-90). In some instances, an anchor-based method approach may be taken to establish the empirical thresholds for meaningful change. For example, an analysis can utitlize pooled, longitudinal data using the following clinical anchors:
PGIC: Worsening in global impression of change.
PGIS: Worsening in global impression of severity.
PGIC: Improvement in global impression of change.
PGIS: Improvement in global impression of severity

The change timepoint and magnitude of worsening and improvement (e.g., ≥1 level change) may be empirically selected based on sample size and distribution of PGIC and PGIS scores.

### B. Determine Effectiveness of Treatment as Compared to Control

In yet another aspect, the disclosure provides a method for assessing treatment effectiveness for an anorectal disorder, the method comprising: receiving, via an input device, a first set of attributes associated with a first plurality of patients receiving a first treatment for an anorectal disorder and a second set of attributes associated receiving a second treatment for the anorectal disorder; automatically inputting, via a processor, the first set of attributes and the second set of attributes to a model, wherein the model is configured to output a first score corresponding to the severity of the anorectal disorder for the first plurality of patients and a second score corresponding to the severity of the anorectal disorder for the second plurality of patients; outputting the first score and the second score, or a representation thereof, via an output device; determining the effectiveness of the first treatment relative to the second treatment based on the first score and the second score. In some embodiments, the first treatment is determined to more effective than the second treatment if the first score is lower than the second score. In some embodiments, the first treatment is determined to be an effective treatment for the anorectal disorder if the first score is higher than the second score.

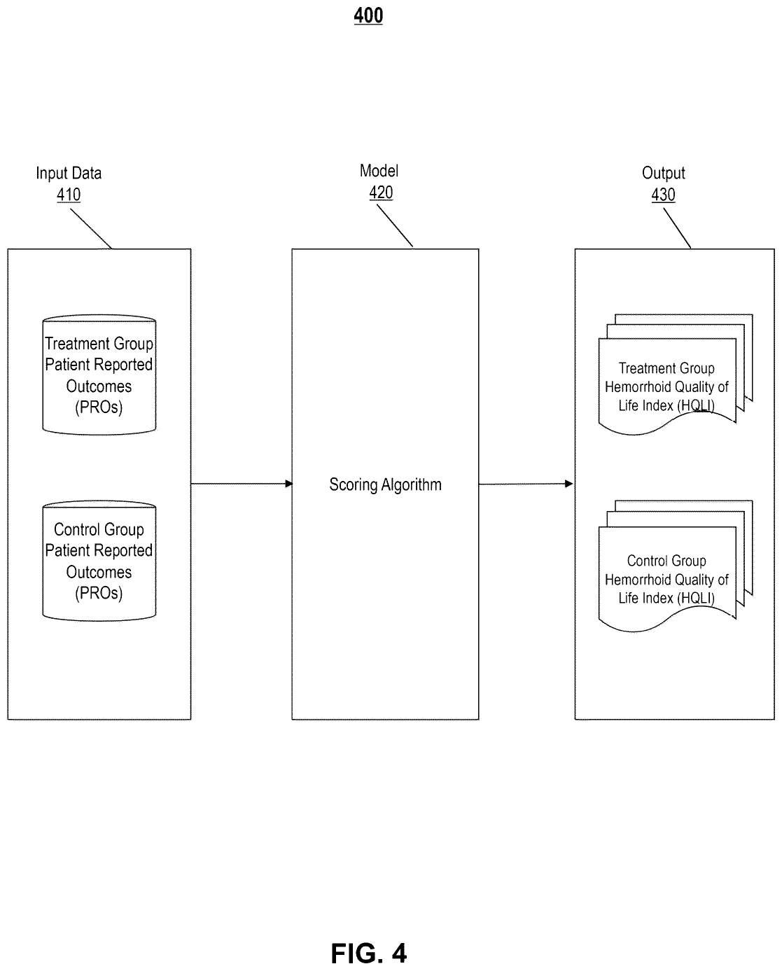

In certain examples, the second treatment is a control or placebo treatment. FIG. 4 illustrates a diagram of such an exemplary process for evaluating the effect of a treatment for an anorectal disorder on a treatment group versus a control group according to one embodiment of the disclosure. Generally, the input data 410 may contain a set of treatment group PRO data as well as a set of control group patient PRO data; the input is passed onto the scoring algorithm model 420, which is configured to output a score corresponding to the severity of the anorectal disorder of the treatment group as well as a score corresponding to the severity of the anorectal disorder of the control group 330. The process may further contain a step of comparing the treatment group score with the control treatment group to determine the effectiveness of the treatment.

In some variations of the method, the input data comprises PRO data from patients receiving two different treatments and the model outputs two scores representing the presence or severity of the anorectal disorder of the two different treatments. The two scores may be compared to compare the effectiveness of the two treatments.

In some embodiments that may be combined with any one of the embodiments above, the anorectal disorder is selected from the group consisting of a hemorrhoid, an anal wart, an anal fissure, an anorectal abscess, and an anal fistula. In some embodiments, the anorectal disorder is a hemorrhoid. In some embodiments, the hemorrhoid is a Goligher Grade II hemorrhoid. In some embodiments, the hemorrhoid is a Goligher Grade III hemorrhoid.

### IV. Computer Software and Systems

### A. Computer Readable Media (Software)

Any of the aforementioned methods of present disclosure may be implemented as computer program processes that are specified as a set of instructions recorded on a non-transitory computer-readable storage medium (also referred to as a computer-readable medium-CRM).

Further provided herein is a non-transitory computer-readable storage medium storing one or more programs, the one or more programs comprising instructions, which when executed by one or more processors of an electronic device having a display, cause the electronic device to perform the method of any one of the preceding embodiments.

Accordingly, in still another aspect, the present disclosure provides a non-transitory computer-readable storage medium storing one or more programs, the one or more programs comprising instructions, which when executed by one or more processors of an electronic device having a display, cause the electronic device to: receive, via an input device, a set of attributes associated with a patient having or suspected having an anorectal disorder; automatically input, via a processor, the set of attributes to a model, wherein the model is configured to output a score corresponding to the severity of the anorectal disorder the patient; and output the score via an output device.

Examples of computer-readable storage media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, ultra-density optical discs, any other optical or magnetic media, and floppy disks. In some embodiments, the computer-readable storage medium is a solid-state device, a hard disk, a CD-ROM, or any other non-volatile computer-readable storage medium.

The computer-readable storage media can store a set of computer-executable instructions (e.g., a “computer program”) that is executable by at least one processing unit and includes sets of instructions for performing various operations.

A computer program (also known as a program, software, software application, script, or code) can be written in any form of programming language, including compiled or interpreted languages, declarative or procedural languages, and it can be deployed in any form, including as a standalone program or as a module, component, or subroutine, object, or other component suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms or portions of code). A computer program can be deployed to be executed on one computer or on multiple computers that are located at one site or distributed across multiple sites and interconnected by a communication network. Examples of computer programs or computer code include machine code, such as is produced by a compiler, and files including higher-level code that are executed by a computer, an electronic component, or a microprocessor using an interpreter.

As used herein, the term “software” is meant to include firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Any combination of separate programs that together implement a software aspect described here is within the scope of the subject disclosure. In some implementations, the software programs, when installed to operate on one or more electronic systems, define one or more specific machine implementations that execute and perform the operations of the software programs.

Any suitable machine learning models may be used with the methods of the present invention and be implemented as computer program processes that are specified as a set of instructions recorded on a computer-readable storage medium. In some embodiments, the model is a discriminative model or a generative model.

### B. Electronic Devices

Further, any one of the preceding methods of the present disclosure may be implemented in one or more computer systems or other forms of apparatus. Examples of apparatus include but are not limited to, a computer, a tablet personal computer, a personal digital assistant, and a cellular telephone.

Accordingly, further provided is an electronic device, comprising: a display; one or more processors; a memory; and one or more programs, wherein the one or more programs are stored in the memory and configured to be executed by the one or more processors, the one or more programs including instructions for performing the method of any one of the preceding embodiments.

In some embodiments, the disclosure further provides an electronic device, comprising: a display; one or more processors; a memory; and one or more programs, wherein the one or more programs are stored in the memory and configured to be executed by the one or more processors, the one or more programs including instructions for receiving, via an input device, a set of attributes associated with a patient having or suspected having an anorectal disorder; automatically inputting, via a processor, the set of attributes to a model, wherein the model is configured to output a score corresponding to the severity of the anorectal disorder the patient; and outputting the score via an output device.

In some embodiments, the electronic device may be a server computer, a client computer, a personal computer (PC), a user device, a tablet PC, a laptop computer, a personal digital assistant (PDA), a cellular telephone, or any machine capable of executing a set of instructions, sequential or otherwise, that specify actions to be taken by that machine. In some embodiments, the electronic device may further include keyboard and pointing devices, touch devices, display devices, and network devices.

As used herein, the terms “computer”, “processor”, and “memory” all refer to electronic or other technological devices. These terms exclude people or groups of people. For the purposes of the specification, the terms “display” or “displaying” means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device described herein for displaying information to the user and a virtual or physical keyboard and a pointing device, such as a finger, pencil, mouse or a trackball, by which the user can provide input to the computer. Other kinds of devices can be used to provide for interaction with a user as well; for example, feedback provided to the user can be any form of sensory feedback, such as visual feedback, auditory feedback, or tactile feedback; and input from the user can be received in any form, including acoustic, speed, or tactile input.

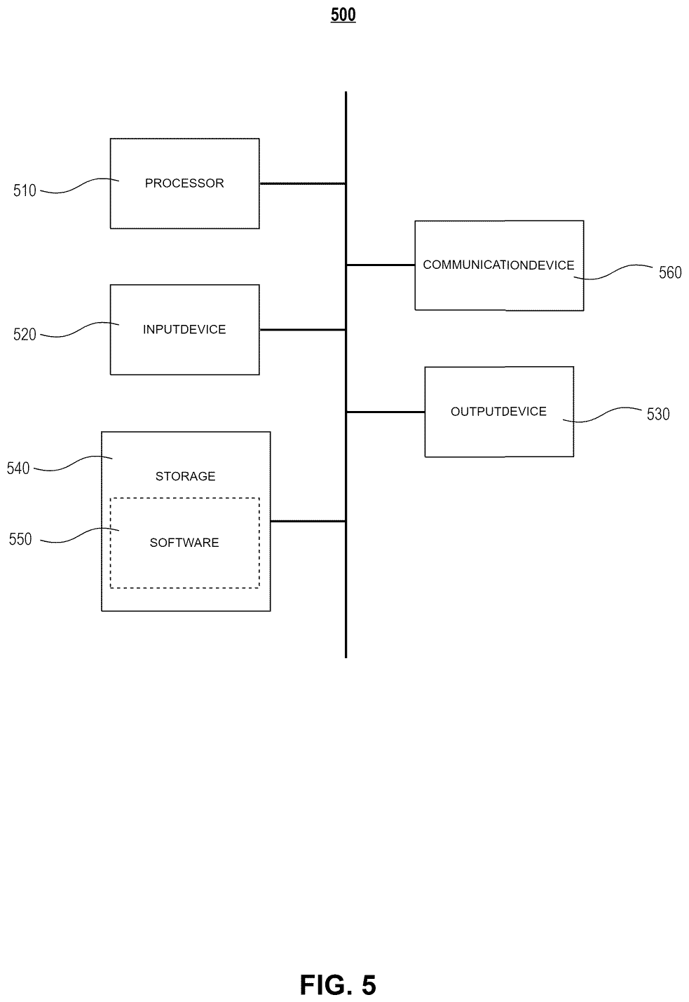

FIG. 5 illustrates an example of a computing device 500 in accordance with one embodiment. Device 500 can be a host computer connected to a network. Device 500 can be a client computer or a server. As shown in FIG. 5, device 500 can be any suitable type of microprocessor-based device, such as a personal computer, workstation, server or handheld computing device (portable electronic device) such as a phone or tablet. The device can include, for example, one or more of processor 510, input device 520, output device 530, storage 540, and a communication device 560. Input device 520 and output device 530 can generally correspond to those described above and can be connectable or integrated with the computer.

Input device 520 can be any suitable device that provides input, such as a touch screen, keyboard or keypad, mouse, or voice-recognition device. Output device 530 can be any suitable device that provides output, such as a touch screen, haptics device, or speaker.

Storage 540 can be any suitable device that provides storage, such as an electrical, magnetic or optical memory including a RAM, cache, hard drive, or removable storage disk. Communication device 560 can include any suitable device capable of transmitting and receiving signals over a network, such as a network interface chip or device. The components of the computer can be connected in any suitable manner, such as via a physical bus or wirelessly.

Software 550, which can be stored in storage 540 and executed by processor 510, can include, for example, the programming that embodies the functionality of the present disclosure (e.g., as embodied in the devices as described above).

Software 550 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 540, that can contain or store programming for use by or in connection with an instruction execution system, apparatus, or device.

Software 550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 500 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 500 can implement any operating system suitable for operating on the network. Software 550 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Example 1. Hemorrhoid Quality of Life Index (HQLI) as Instrument for Evaluating Treatment Effectiveness of Grades II and III Hemorrhoids in Clinical Trial

This example demonstrates the use of an exemplary Hemorrhoid Quality of Life Index (HQLI) as an instrument to evaluate treatment effectiveness for Grades II and III hemorrhoids in clinical trial.

### Hemorrhoid Quality of Life Index (HQLI)

The exemplary HQLI includes two parts. Part I comprises a set of five hemorrhoidal disease symptom intensity items, intended to be assessed daily with a 24-hour recall period. These items employ an 11-point numerical rating scale (NRS) with anchors at 0 for no (symptom), 5 for moderate (symptom), and 10 for worst possible (symptom). Part II includes ten items that incorporate a 7-day recall period for weekly administration. These items utilize five-category verbal response scales ranging from not at all [impact] to extremely or a great deal [impact].

There are five domains included in the HQLI: Hemorrhoid Symptom Intensity, Hemorrhoidal Bleeding, Hemorrhoidal Bother, Daily Activity Impacts, and Emotional Impacts. Symptom Impact is aggregated to Physical and Mental.

### Clinical Trial

CITI-002 (lidocaine hydrochloride (HCl) and halobetasol propionate) cream is being developed as a fixed-dosed combination (FDC) product for the topical treatment and symptomatic relief of hemorrhoids.

This phase 2 trial is a multi-center, randomized, double-blind, parallel-group comparison of two different CITI-002 cream strengths versus its monads. Qualifying subjects presenting with Grade II or III hemorrhoids were randomized to treatment applied twice daily to the peri-rectal area as well as the distal aspect of the anal canal using the product applicator tip.

The treatment arms were as follows:
Lidocaine HCl cream, 5.78%
Halobetasol propionate cream, 0.02%

Halobetasol propionate cream, 0.05%
CITI-002 (lidocaine HCl and halobetasol propionate) cream, 5.78%/0.02%
CITI-002 (lidocaine HCl and halobetasol propionate) cream, 5.78%/0.05%

The total study duration was approximately 45 days, i.e., up to 30 days for screening, one week (7 days) of treatment, plus an additional 7 days for safety follow up. Table 1 below shows the clinical outcome assessment (COA) schedule.

TABLE 1
Clinical Outcome Assessment (COA) Schedule

| | Visit | | | | |
|---|---|---|---|---|---|
| | Visit 1 | Visit 2 | Visit 3 | Visit 4 | Visit 5 |
| | Screening/ | Follow- | Follow- | End of | End of Study |
| | Baseline¹ | up 1 | up 2 | Treatment | (By Phone) |
| Days | 1 | 2 | 5 | 8 ± 1 | 15 + 2 |
| Informed Consent | X | | | | |
| Pain and Burning Screening (Modified HQLI Question 1 & 2) | X² | | | | |
| Hemorrhoid Quality of Life Index (HQLI): Part I³ | X | | | X | X |
| Hemorrhoid Quality of Life Index (HQLI): Part II | X | | | X | X |
| Hemorrhoid Severity Score (HSS) | X | | | X | |
| NIH Fear Affect (NIHFA) | X | | | X | |
| PROMIS Pain Interference | X | | | X | |
| Patient Global Impression of Severity (PGIS) | X | | | X | X |
| Patient Global Impression of Change (PGIC) | | | | X | X |

¹Subjects can be screened up to 30 days prior to Baseline (Day 1). Subjects who require washout for longer than 30 days will be reconsented and screening procedures redone.
²Pain and burning ratings will be assessed by the subject for their symptoms at the time of the visit via a paper questionnaire modified from the HQLI Questions 1 and 2. Ratings on pain and/or burning must be ≥6 at the time of randomization to be eligible for the trial.
³The HQLI Part I will be completed by subjects every day from Visit 1 though Visit 5, typically at approximately the same time every day, after dinner and prior to bedtime; at Visits 4 and 5, HQLI Part I will be completed during the visit.

### Phase 2 Study Endpoints

The primary endpoint for the phase 2 trial was a reduction in hemorrhoidal disease symptoms (HQLI Part I: pain, burning, itching, swelling, bleeding).

The secondary endpoint for the phase 2 trial was the reduction in hemorrhoidal disease symptom physical impact (HQLI Part II: bowel movement (BM) interference, pressure in bowels).

HQLI exploratory endpoints for the phase 2 trial included reduction in hemorrhoidal disease symptom impact on daily activities and emotional function (HQLI Part II: daily activity, emotional impact).

### HQLI Scoring

The HQLI was scored according to the component parts. Part I items were administered daily by electronic data capture (EDC). The five individual daily symptom item responses were retained so that within-week change trajectories could be analyzed. Item data was aggregated weekly by taking the mean response across seven days, subject to the missing data rules.

Part II items were administered weekly by EDC with a 7-day recall period. The individual item weekly data was retained.

The scoring procedures are summarized below:

Individual items were combined into scales, pending acceptable evidence for internal consistency reliability and construct validity. Preliminary scaling evidence indicated that the HQLI data can be combined into five scales and one symptom item. Part I items 1-5 (pain, burning, itching, swelling, bleeding) were combined in the Hemorrhoidal Disease (HD) Symptom Intensity Summary Scale. The mean of the component items were calculated and retained as the daily score, subject to the missing data rules. Daily scale scores were aggregated as the mean across 7 days, again subject to missing data rules. Daily scale scores were important since the active treatment period was seven days.

### HQLI Part I Symptom Intensity Summary (SIS) Score

1. Calculate the mean of Items 1-5 for each day in the assessment week to produce Daily SIS score.
2. Calculate the mean Daily SIS scores of the valid days across the seven days in the assessment week to produce the Weekly SIS score.

Preliminary scaling evidence indicated that Part II can be scored as three domain, two summary, and a total score. Items 7 and 8 (comfortable bowel movement & pressure) were combined to form the Hemorrhoidal Bother scale by calculating the mean score of the two items. Daily Activity Impact was scored as the mean of items 9, 11, and 12 (daily activities, physical activities, sitting). The Emotional Impact domain scale was scored as the mean of items 6, 10, 13, 14, and 15 (discomfort, social activities, anxiety, embarrassment, and frustration). Hemorrhoidal Bother and Daily Activity Impact was combined to form the Physical Summary score by calculating the mean of the two scales. The mean, rather than the total, was used to preserve the original item ratings to aid in interpretation. The Physical Summary was combined with the Emotional Impact (Mental) scale by calculating the mean score to form the Symptom Impact Total score.

The scoring procedures are summarized below, pending application of the missing data rules.

### HQLI Part II Symptom Impact Score (by Domain)

### Hemorrhoidal Bother Scale

Calculate the mean of Items 7 and 8 on day 7 of each assessment week (i.e., Study Days 8 and 15) to produce the weekly Hemorrhoidal Bother score.

### Daily Activity Impact Scale

Calculate the mean of Items 9, 11, and 12 on day 7 of each assessment week (i.e., Study Days 8 and 15) to produce the weekly Daily Activity Impact score.

### Emotional Impact Scale

Calculate the mean of Items 6, 10, 13, 14, and 15 on day 7 of each assessment week (i.e., Study Days 8 and 15) to produce the weekly Emotional Impact score.

### HQLI Part II Summary Score

### HQLI Physical Summary Scale

Calculate the mean of the Hemorrhoidal Bother and Daily Activity Impact scale scores of each assessment week (i.e., Study Days 8 and 15) to produce the weekly Physical Summary score.

### HQLI Mental Summary Scale

The HQLI Mental Summary Scale score is equal to the Emotional Impact scale score. The label is utilized for descriptive purposes that are consistent with other PRO instruments that utilize the Physical/Mental dichotomy.

### HQLI Part II Symptom Impact Total Score

Calculate the mean of Part II Items 6-15 of each assessment week (i.e., Study Days 8 and 15) to produce the weekly HQLI Symptom Total Impact score.

### Example 2. Analysis of Treatment Effectiveness from Clinical Trial Top Line Results

The efficacy and safety of delivering the pharmaceutical composition comprising Lidocaine HCl and Halobetasol Propionate (CITI-002) towards topical treatment and symptomatic relief of hemorrhoids was studied on a patient population in a clinical trial. This example demonstrates the use of Hemorrhoid Quality of Life Index (HQLI) as an instrument in evaluating the treatment effectiveness using top line results from the clinical trial CITI-002.

This trial was a multi-center, randomized, double-blind, parallel-group comparison of CITI-002 versus its monads. Qualifying subjects presenting with Grade II or III hemorrhoids were randomized to treatment applied twice daily to the peri-rectal area as well as the distal aspect of the anal canal using the product applicator tip.

The treatment groups were as follows:
A single therapy cream containing a topical anesthetic, Lidocaine HCl, 5.78%.
A single therapy cream containing a topical corticosteroid, Halobetasol Propionate (HBP), high strength (0.05%).
A combination cream containing Lidocaine HCl and Halobetasol Propionate (5.78%/0.05%)-CITI-002.

### Efficacy Results

### Reduction in Symptoms

As shown in Tables 2-6, CITI-002 had a greater treatment effect at Day 8 compared to the component monad treatments for SIS (Table 2), Pain (Table 3), Burning (Table 4), Swelling (Table 5), and Bleeding (Table 6).

This greater treatment effect at the end of the treatment across these four individual symptoms, as well as the Symptom Intensity Summary (SIS) Score, shows that the formulation has a greater effect than its component monad ingredients in the modified intention-to-treat (mITT) population.

TABLE 2
SIS Success by MCT (Reduction ≥5.247 points) at Day 8 (mITT).

| Treatment Arm | Symptom Intensity Summary (SIS) (%) |
|---|---|
| CITI-002 | 42 |
| HBP 0.05% | 29 |
| Lidocaine | 21 |

TABLE 3
Pain Success by MCT (Reduction ≥6 points) at Day 8 (mITT).

| Treatment Arm | Symptom Intensity Summary (SIS) (%) |
|---|---|
| CITI-002 | 42 |
| HBP 0.05% | 31 |
| Lidocaine | 31 |

TABLE 4
Burning Success by MCT (Reduction ≥6 points) at Day 8 (mITT).

| Treatment Arm | Symptom Intensity Summary (SIS) (%) |
|---|---|
| CITI-002 | 42 |
| HBP 0.05% | 39 |
| Lidocaine | 30 |

TABLE 5
Swelling Success by MCT (Reduction ≥5 points) at Day 8 (mITT).

| Treatment Arm | Symptom Intensity Summary (SIS) (%) |
|---|---|
| CITI-002 High | 48 |
| HBP 0.05% | 42 |
| Lidocaine | 39 |

TABLE 6
Bleeding Success by MCT (Reduction ≥3
points) at Day 8 (mITT).

| Treatment Arm | Symptom Intensity Summary (SIS) (%) |
|---|---|
| CITI-002 | 50 |
| HBP 0.05% | 48 |
| Lidocaine | 44 |

Moreover, proportionally more patients in the CITI-002 cohort reported meaningful and statistically significant improvement as compared to patients treated with lidocaine alone (CMH test, p=0.035).

Taken together, the clinical trial top line results show that CITI-002 is more effective than its individual components as a treatment for hemorrhoids, and this example demonstrates that HQLI is a valid and robust instrument in evaluating the treatment effectiveness using top line results from the clinical trial CITI-002.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for assessing an anorectal disorder in a patient, the method comprising:
(a) receiving, via an input device, a set of attributes associated with a patient having or suspected having an anorectal disorder;
(b) automatically inputting, via a processor, the set of attributes to a model, wherein the model is configured to output a score corresponding to the severity of the anorectal disorder the patient; and
(c) outputting the score via an output device.

2. The method of claim 1, wherein the anorectal disorder is selected from the group consisting of a hemorrhoid, an anal wart, an anal fissure, an anorectal abscess, and an anal fistula.

3. The method of claim 2, wherein the anorectal disorder is a hemorrhoid.

4. The method of claim 3, wherein the hemorrhoid is a Goligher Grade II hemorrhoid.

5. The method of claim 3, wherein the hemorrhoid is a Goligher Grade III hemorrhoid.

6. The method of any one of claims 1-5, wherein the set of attributes comprise one or more symptoms of the patient.

7. The method of claim 6, wherein the one or more symptoms comprise pain, burning, itching, swelling, and/or bleeding.

8. The method of any one of claims 1-7, wherein the set of attributes comprise a symptom intensity and/or a symptom impact for the one or more symptoms of the patient.

9. The method of claim 8, wherein the symptom intensity is collected daily.

10. The method of claim 8, wherein the symptom impact is collected weekly.

11. The method of claim 8, wherein the symptom impact comprises a physical summary and/or a mental summary.

12. The method of claim 11, wherein the physical summary comprises an anorectal disorder bother and/or a daily activity impact, and wherein the mental summary comprises an emotional impact.

13. The method of claim 12, wherein the anorectal disorder bother comprises a bowel movement interference and/or a bowel pressure, wherein the daily activity impact comprises a normal daily activity impact, an exercise or strenuous activity impact, and/or a sitting impact, and wherein the emotional impact comprises a psychological distress impact, a discomfort impact, and/or a social impact.

14. The method of any one of claims 1-13, wherein the set of attributes comprise one or more demographic factors selected from the group consisting of age, gender, race, ethnicity, income, education, and occupation.

15. The method of any one of claims 1-14, wherein the set of attributes comprises one or more attributes from a patient reported outcome (PRO), a clinician interview, and/or a cognitive interview.

16. The method of claim 8, wherein the symptom intensity and/or the symptom impact are represented in a numerical rating scale.

17. The method of claim 16, wherein the symptom intensity is represented in an 11-point numerical rating scale of 0 to 10, wherein a 0 represents no intensity (no symptom), a 5 represents moderate intensity (moderate symptom), and a 10 represents highest intensity (worst symptom).

18. The method of claim 16, wherein the symptom impact is represented in a 5-point numerical rating scale of 0 to 4, wherein a 0 represents no impact and a 4 represents highest impact.

19. The method of any one of claims 1-18, wherein the model is a trained model using a training method selected from the group consisting of 2-class learning classification and regression technique comprising: logistic regression, 2-class SVM, nearest neighbor, decision tree, neural net, random forest, deep learning, support vector regression, linear regression, non-linear regression, multivariate regression, robust regression, neural network regression, random forest regression, and nearest neighbors; a 1-class learning technique comprising: 1-class SVM, minimum enclosing ball, clustering-based anomaly detection, nearest neighbor-based anomaly detection; and a 1.5-class learning technique comprising: joint optimization, transfer, multi-task, and low C-value formulations.

20. The method of any one of claims 1-19, wherein the model has been tested for test-retest reliability and/or internal consistency reliability.

21. The method of any one of claims 1-20, wherein the model has been validated for known-groups validity and/or convergent validity.

22. The method of any one of claims 1-21, wherein the output score comprises a symptom intensity score and a symptom impact score.

23. The method of claim 22, wherein the symptom intensity score is a daily score.

24. The method of claim 22, wherein the symptom impact score is a weekly score.

25. The method of any one of claims 22-24, wherein the symptom impact score comprises a physical summary score and a mental summary score.

26. The method of any one of claims 22-25, wherein the symptom impact score comprises an anorectal disorder bother score, a daily activity impact score, and an emotional impact score.

27. The method of any one of claims 1-26, further comprising a step of comparing the output score with a predetermined threshold value.

28. The method of any one of claims 1-27, further comprising a step of diagnosing the presence and/or severity of the anorectal disorder of the patient based on the output score.

29. The method of any one of claims 1-28, further comprising a step of selecting one or more treatment options for the patient based on the output score.

30. A method for assessing treatment effectiveness for an anorectal disorder, the method comprising:
(a) recording a first set of attributes associated with a patient prior to a treatment and a second set of attributes associated with the patient during or after the treatment, wherein the patient suffers from an anorectal disorder;
(b) inputting, via an input device, the first set of attributes associated with the patient prior to a treatment and the second set of attributes associated with the patient during or after the treatment;
(c) automatically inputting, via a processor, the first set of attributes and the second set of attributes to a model, wherein the model is configured to output a first score corresponding to the severity of the anorectal disorder of the patient prior to the treatment and a second score corresponding to the severity of the anorectal disorder of the patient during or after the treatment;
(d) outputting the first score and the second score, or a representation thereof, via an output device; and
(e) determining the effectiveness of the treatment based on the first score and the second score.

31. The method of claim 30, wherein the recording of step (a) and inputting of step (b) occur simultaneously.

32. The method of claim 30 or 31, wherein the recording of step (a) is performed on paper.

33. The method of any one of claims 30-32, wherein the treatment is determined to be effective treatment for the anorectal disorder if the first score is lower or higher than the second score.

34. The method of any one of claims 30-33, wherein the first score and the second score are symptom intensity summary (SIS) scores.

35. The method of any one of claims 30-34, wherein the treatment is determined to be effective treatment for the anorectal disorder if (1) the first score is lower or higher than the second score; and (2) if the difference between the first score and the second score is greater than a predetermined meaningful change threshold (MCT).

36. A method for assessing treatment effectiveness for an anorectal disorder, the method comprising:
(a) receiving, via an input device, a first set of attributes associated with a first plurality of patients receiving a first treatment for an anorectal disorder and a second set of attributes associated with a second plurality of patients receiving a second treatment for the anorectal disorder;
(b) automatically inputting, via a processor, the first set of attributes and the second set of attributes to a model, wherein the model is configured to output a first score corresponding to the severity of the anorectal disorder for the first plurality of patients and a second score corresponding to the severity of the anorectal disorder for the second plurality of patients;
(c) outputting the first score and the second score, or a representation thereof, via an output device; and
(d) determining the effectiveness of the first treatment relative to the second treatment based on the first score and the second score.

37. The method of claim 36, wherein the first treatment is determined to more effective than the second treatment if the first score is lower or higher than the second score.

38. The method of any one of claims 36-37, wherein the first treatment or the second treatment is a control or placebo treatment.

39. The method of claim 38, wherein the first treatment is determined to be an effective treatment for the anorectal disorder if the first score is lower or higher than the second score.

40. The method of any one of claims 30-39, wherein the anorectal disorder is selected from the group consisting of a hemorrhoid, an anal wart, an anal fissure, an anorectal abscess, and an anal fistula.

41. The method of any one of claims 30-40, wherein the anorectal disorder is a hemorrhoid.

42. The method of claim 41, wherein the hemorrhoid is a Goligher Grade II hemorrhoid.

43. The method of claim 42, wherein the hemorrhoid is a Goligher Grade III hemorrhoid.

44. A non-transitory computer-readable storage medium storing one or more programs, the one or more programs comprising instructions, which when executed by one or more processors of an electronic device having a display, cause the electronic device to perform the method of any one of claims 1-43.

45. An electronic device, comprising:
a display;
one or more processors;
a memory; and
one or more programs, wherein the one or more programs are stored in the memory and configured to be executed by the one or more processors, the one or more programs including instructions for performing the method of any one of claims 1-43.
